(19) United States
(12) Patent Application Publication
(10) Pub. No.: US 2007/0281905 A1
(43) Pub. Date: Dec. 6, 2007

(54) USE OF SIMETHICONE IN CONSTIPATED PATIENTS

(76) Inventors: Martina GRIPP, Ingelheim (DE); Christoffel SCHUIJT, Hofheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/549,392

(22) Filed: Oct. 13, 2006

Related U.S. Application Data
(63) Continuation of application No. PCT/EP05/03795, filed on Apr. 12, 2005.

(30) Foreign Application Priority Data
Apr. 13, 2004 (EP) 04 008 736

(51) Int. Cl. A61K 31/695

(52) U.S. Cl. 514/63

(57) ABSTRACT

The invention relates to the use of simethicone for the preparation of a medication for the treatment of constipated persons suffering from bloated feeling during the night.

### BACKGROUND OF THE INVENTION

1. Technical Field

The invention relates to the use of simethicone for the preparation of a medication for the treatment of constipated persons suffering from bloated feeling.

2. Description of the Prior Art

Constipation is suffered by a considerable number of people. Stomach pressure and bloating is the most suffered accompanying symptom world-wide. Approximately 20% of world wide population suffers from constipation. Some causes are fully known, such as several illnesses and certain medications; other causes are mentioned and broadly accepted, such as food (little fibre intake, not enough liquids and un-regular meals), lifestyle symptoms (stress, little exercise), gender and age.

Dimethicone is a well known pharmaceutical material consisting of linear siloxane polymers containing repeating units of the formula {—(CH₂)₂SiO}ₙ stabilized with trimethylsiloxy end blocking units of the formula [(CH₃)₃SiO—].

Simethicone is the mixture of dimethicone and silicon dioxide. It is well known that simethicone can be used for the relief of flatulence and similar discomforts.

Laxative agents include bisacodyl, sodium picosulphate, cascara sagrada, danthron, senna, phenolphthalein, aloe, castor oil, ricinoleic acid, and dehydrocholic acid and mixtures of these laxatives, as well as certain polyethylene glycols (macrogols), lactulose, sorbitol, glycerin, parafine, sodium sulphate and magnesium sulphate, of which bisacodyl, sodium picosulphate and macrogol 3350 are preferred.

The French patent application FR 2 828 105 suggests that antiflatulents such as simethicone prevent the side effects of poorly absorbed polysaccharides in the treatment or prevention of constipation.

U.S. Pat. No. 6,676,933 discloses a pharmaceutical composition comprising mosapride, pancreatin, and simethicone or dimethicone for the treatment of gastrointestinal disorders such as indigestion, constipation and flatulence.

The International patent application WO 95/01803 suggests a pharmaceutical composition comprising famotidine for the treatment of gastrointestinal disorders such as indigestion, constipation and optionally simethicone to relieve flatulence.

The European patent application EP 1 297 825 suggests a composition comprising simethicone and a adsorbant in a ratio of at least 1:2.22, furthermore a combination with other active ingredients such as bisacodyl is suggested.

The product PURGAZEN® of AGEFA GmbH, Austria (http://aposhop.aponet.at/OTCkatalog/htm/purgazen_dragees.htm) contains a combination of 5 mg bisacodyl and 10 mg of dimeticon.

The European patent application EP 1 086 701 A suggests a composition comprising a laxative, in particular bisacodyl or senna; and simethicone for enhancing the efficacy of the laxative.

There is still a high demand for a medication which allows an immediate effect of reducing the bloated feeling and an overnight relief of constipation in constipated persons.

### BRIEF DESCRIPTION OF THE INVENTION

It has been found surprisingly that simethicone provides an immediate effect on bloated feeling, gas discomfort and flatulence in constipated persons, and that especially the combination with bisacodyl achieves an overnight relief of constipation with considerably less flatulence.

Accordingly the invention relates to the use of simethicone for the preparation of a medication for the treatment of constipated persons suffering from bloated feeling during the night.

Moreover, the invention relates to a pharmaceutical composition comprising 80 to 300 mg, in particular 90 to 220 mg, most preferably about 210 mg of simethicone, 2 to 20 mg of bisacodyl and one or more pharmaceutically acceptable carriers and/or auxiliaries, in a way that simethicone reduces the bloated feeling and the flatulence without enhancing the efficacy of bisacodyl against constipation.

A further aspect of the invention is a kit of parts for the preparation of a medication for the treatment of constipated persons suffering from bloated feeling comprising at least two compartments wherein
(a) one compartment comprises 80 to 300 mg, in particular 90 to 220 mg, most preferably about 210 mg of simethicone and a pharmaceutically acceptable carrier and/or auxiliary,
(b) the other compartment comprises 2 to 20 mg of bisacodyl and one or more pharmaceutically acceptable carrier and/or auxiliary.

Moreover, the invention relates to a method of treating the bloated feeling of constipated persons during the night, which method comprises administering a therapeutically effective amount of simethicone to said constipated persons.

Finally, the invention relates to an article of manufacture comprising packaging material contained within which is the composition effective to treat the bloated feeling of a constipated person according to this invention and the packaging material comprises a label which indicates that the composition can be used to treat the bloated feeling and gas discomfort of a constipated person.

### BRIEF DESCRIPTION OF THE DRAWINGS

FIG. 1 shows the mean scores of feeling of bloatedness depending on the time after administration until defecation of

| | |
|---|---|
| —♦— | 10 mg of bisacodyl; |
| —▴— | 210 mg of simethicone; |
| —▪— | 10 mg of bisacodyl + 210 mg of simethicone. |

FIG. 2 shows the change from baseline in mean scores of feeling of bloatedness depending on the time after administration until defecation of

| | |
|---|---|
| —♦— | 10 mg of bisacodyl; |
| —▴— | 210 mg of simethicone; |
| —▪— | 10 mg of bisacodyl + 210 mg of simethicone. |

FIG. 3 shows the global assessment of efficacy by patients regarding the feeling of bloatedness after administration of 10 mg of bisacodyl (BS 10 mg), 210 mg of simethicone (SM210 mg) or 10 mg of bisacodyl+210 mg of simethicone (BS 10 mg+SM210 mg)

FIG. 4 shows the global assessment of efficacy by patients regarding the constipation after administration of 10 mg of bisacodyl (BS 10 mg), 210 mg of simethicone (SM210 mg) or 10 mg of bisacodyl+210 mg of simethicone (BS 10 mg+SM210 mg).

### DETAILLED DESCRIPTION OF THE INVENTION

In a preferred embodiment simethicone is used for the treatment of constipated persons, who suffer from bloated feeling during the night.

The level of simethicone in the present invention to reduce bloated feeling in constipated persons is generally from about 80 mg to about 300 mg, preferably from about 90 to about 250 mg, in particular from about 100 mg to about 220 mg, most preferably about 210 mg per administration. A dosage unit can be one tablet, capsule, or suppository, one teaspoonful of a liquid, or one single portion of any other suitable delivery form.

The phrase “constipated person” as used hereinbefore or herein below is intended to mean a person, who suffers from constipation due to illnesses, certain medications; food in-take, lifestyle exercise, gender or age, or any other possible reason. As a rule such a person may be male or female, predominately female, of any age including children, young adults, adults and elder persons, preferably persons in the range of 40 to 80, in particular 45 to 70 years are affected by severe constipation. As a rule the constipated person will not be treated with poorly absorbed polysaccharides.

The phrase “combination therapy” (or “co-therapy”), in defining use of simethicone and a laxative, is intended to embrace administration of each agent in a sequential manner in a regimen that will provide beneficial effects, in particular reduction of the bloated feeling during the night and defecation the following morning, of the drug combination. The phrase also is intended to embrace co-administration of these agents in a substantially simultaneous manner, such as in a single capsule having a fixed ratio of these active agents or in multiple, separate capsules for each agent; in this case the simethicone can be taken any time of the day, so that the bloatedness can be treated with immediate effect as it occurs.

The phrase “therapeutically-effective” is intended to qualify the amount of each agent for use in the combination therapy which will achieve the goal of improvement in reduction of bloatedness, gas discomfort, and relief from constipation of each agent by itself, while avoiding adverse side effects typically associated with alternative therapies.

The phrase “immediate effect” with respect to the reduction of the bloated feeling is intended to mean an effect which sets on within 0 to 6 hours, preferably within 1 to 5 hours, in particular about 2 to 3 hours after administration.

The phrase “during the night” or “overnight relief” with respect to the relief of constipation is intended to mean that the defecation takes place 7 to 15 hours, preferably 8 to 14 hours after administration, in particular at the next morning when administered about 0 to 3 hours before bedtime, without any risk of premature defecation.

Preferably the complete dose of simethicone, in particular 80 to 300 mg thereof is administered 1 to 5, in particular 2 to 3 hours before said persons go to bed.

Furthermore preferred is a combination therapy, wherein a laxative selected from the group consisting of bisacodyl, sodium picosulphate, and macrogols is co-administered to the constipated person in need thereof.

The level of laxative is the amount necessary to provide the desired effect, which is generally from about 2.0 mg to about 20 mg, preferably from about 2.5 mg to about 15 mg, and most preferably from about 3.0 mg to about 10.0 mg per dose for bisacodyl.

Most preferably 2 to 20 mg, in particular 3 to 10 mg, of bisacodyl is co-administered with simethicone in a combined form, or separately, or separately and sequentially, wherein the sequential administration is close in time.

The ratio of simethicone to bisacodyl in the compositions according to the invention is, as a rule, in the range of 200:1 to 2:1, preferably in the range 100:1 to 5:1, in particular in the range of about 25:1 to 15:1.

The present invention can be delivered in form of one or more capsules, tablets, chewable tablets, liquid drinks, suppositories, or other pharmaceutically acceptable forms. Oral delivery forms are preferred.

In a preferred embodiment, the invention relates to a pharmaceutical composition comprising two different kinds of granules, one of which is a fast release granule of simethicone and the other is bisacodyl in form of a sustained release granule.

Commonly known pharmaceutically acceptable carriers and/or auxiliaries for orally-administered drugs such as enteric polymers, taste-masking polymers, binders, sweeteners, flavouring agents, dispersants, buffering agents and the like may be included in amounts that do not adversely affect the novel properties of the medication described and claimed herein. Suitable enteric polymer systems include polymethacryaltes (e.g., EUDRAGIT L30D or S100, available from Rohm Company); cellulose acetate phthalate; polyvinyl acetate phthalate, hydroxypropyl methylcellulose phthalate. Suitable binders include microcrystalline cellulose, calcium phosphates, dextrates. Suitable dispersants include croscarmellose sodium, methylcellulose, hydroxymethylcellulose, hydroxypropylmethylcellulose, hydroxyethylcellulose and the like. Suitable sweeteners include sugar, sorbitol, saccharin, mannitol, glucose, aspartame, sucralose and the like. Flavouring agents include peppermint, spearmint, cinnamon, vanilla and the like. A more complete listing of appropriate additives can be found in numerous publications including Remington's Encylcopedia.

Preferred is a kit of parts which comprises
(a) one compartment comprising 80 to 300 mg, preferably 80 to 220 mg, most preferably about 210 mg of simethicone in form of a slow release composition,
(b) one compartment comprising 2 to 20 mg, preferably 3 to 10 mg of bisacodyl in form of a sustained release composition.

### EXAMPLES

The Examples that follow serve to illustrate some formulations according to the invention. They are intended solely as possible procedures described by way of example, without restricting the invention to their content.

Clinical Studies have been carried out in order to compare the influence of simethicone, bisacodyl and the combined doses of simethicone and bisacodyl on the bloated feeling of constipated persons.

The studies were randomized, open and parallel groups. The studies were designed to assess the safety, tolerability, and preliminary efficacy of simethicone and bisacodyl in patients suffering from constipation.

The study was performed with

simethicone 105 mg per capsule,

bisacodyl: 5 mg per tablet

per treatment 2 tablets and/or 2 capsules, respectively

patients were asked to take medication approximately 3 hrs before bedtime.

All patients, including male and female aged 45 to 80 years, participated in an outpatient fashion.

A total of 30 constipated patients, also suffering from bloating participated in these studies.

10 patients were administered with 210 mg of simethicone;

10 patients were administered with 10 mg of bisacodyl; and

10 patients were administered with 210 mg of simethicone and 10 mg of bisacodyl.

Safety assessments included adverse event profile, physical examination, vital sign measurements (defecation, bloating, weight, temperature, heart rate, blood pressure [BP],), clinical laboratory assessments and others.

All safety analyses were conducted on the safety population, defined as all patients who received at least 1 dose of study medication. Descriptive statistics were provided among other things for vital signs and clinical laboratory assessments.

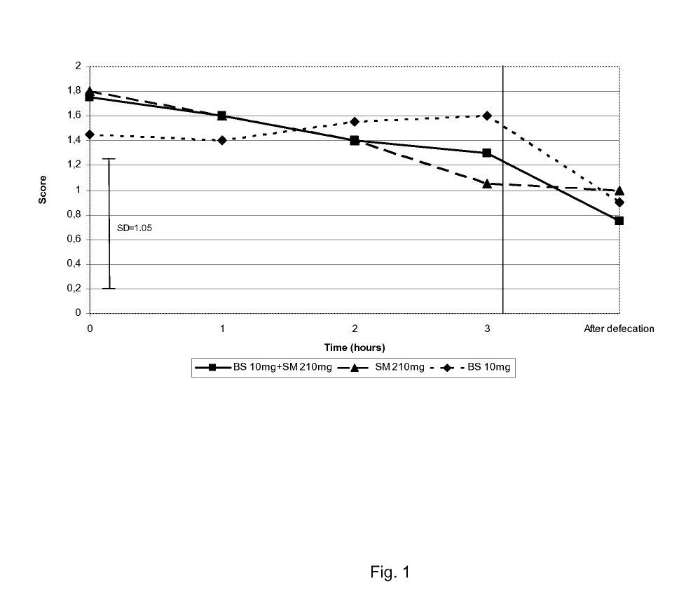

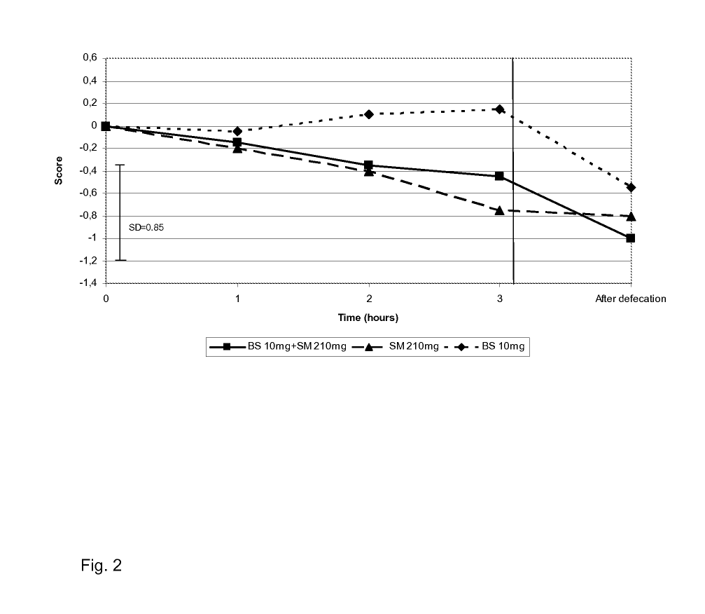

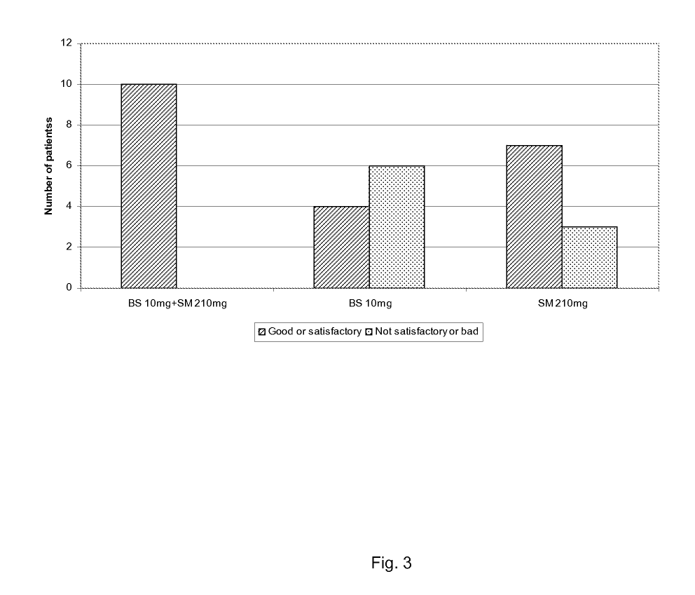

The results regarding the monitoring of the bloated feeling are shown in FIG. 1 to FIG. 3.

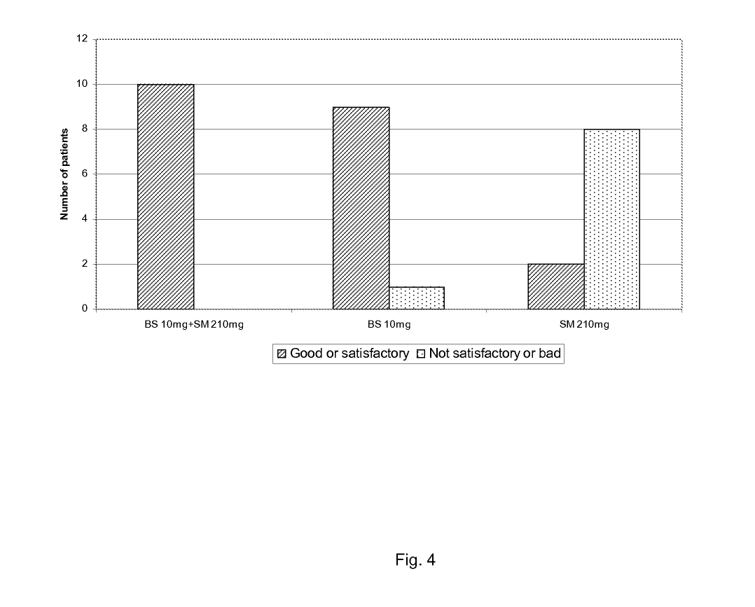

FIG. 4 additionally shows the results regarding constipation.

The results given in these figures clearly show that the treatment of constipated people with simethicone reduces the bloated feeling relatively fast after ingestion. The laxative effect of bisacodyl combined with the direct effect on the bloated feeling by simethicone results in an immediate effect on the bloated feeling and an overnight relief after ingestion in the evening of constipation with considerably less flatulence.

1. A method of treating constipated person suffering from bloated feeling and gas discomfort during the night, the method comprising the step of administering a medicament comprising simethicone to a constipated person suffering from bloated feeling and gas discomfort during the night.

2. The method according to claim 1, wherein the constipated person suffers from bloated feeling during the night.

3. The method according to claim 1, wherein the daily dose of simethicone is from 80 to 300 mg.

4. The method according to claim 1, wherein the daily dose of simethicone is from 90 to 220 mg.

5. The method according to claim 1, wherein the complete dose of simethicone is administered 1 to 5 hours before said person goes to bed.

6. The method according to claim 1, wherein the complete dose of simethicone is administered about 3 hours before said person goes to bed.

7. The method according to claim 1, wherein a laxative is co-administered in a combined form, or separately, or separately and sequentially, wherein the sequential administration is close in time or remote in time.

8. The method according to claim 7, wherein the laxative is selected from the group consisting of bisacodyl, sodium picosulphate, and macrogol.

9. The method according to claim 1, wherein 2 to 20 mg of bisacodyl are co-administered with simethicone in a combined form, or separately or separately and sequentially wherein the sequential administration is close in time.

10. A pharmaceutical composition comprising 80 to 300 mg of simethicone, 2 to 20 mg of bisacodyl, and one or more pharmaceutically acceptable carriers and auxiliaries, wherein the simethicone reduces the bloated feeling without enhancing or reducing the efficacy of bisacodyl against constipation.

11. A composition of claim 10, comprising two different kinds of granules, one of which is a fast release granule of simethicone and the other is bisacodyl in form of a sustained release granule.

12. A kit of parts for the preparation of a medication for the treatment of constipated persons suffering from bloated feeling comprising at least two compartments which comprises
(a) one compartment comprising 80 to 300 mg of simethicone and a pharmaceutically acceptable carrier and/or auxiliary,
(b) one compartment comprising 2 to 20 mg of bisacodyl and one or more pharmaceutically acceptable carrier and/or auxiliary.

13. A kit of parts according to claim 12 which comprises
(a) one compartment comprising 80 to 300 mg of simethicone in form of a immediate release composition,
(b) one compartment comprising 2 to 20 mg of bisacodyl in form of a sustained release composition.
